(19) United States
(12) Patent Application Publication
(10) Pub. No.: US 2026/0193582 A1
(43) Pub. Date: Jul. 9, 2026

(54) FILTER, CELL MEMBRANE DISRUPTION DEVICE, AND CELL MEMBRANE DISRUPTION METHOD

(71) Applicant: Murata Manufacturing Co., Ltd., Nagaokakyo-shi (JP)

(72) Inventors: Masakazu TAKAO, Nagaokakyo-shi (JP); Takashi Kondo, Nagaokakyo-shi (JP); Natsuko Tokitou, Nagaokakyo-shi (JP)

(21) Appl. No.: 19/551,703

(22) Filed: Feb. 27, 2026

Related U.S. Application Data
(63) Continuation of application No. PCT/JP2024/031566, filed on Sep. 3, 2024.

(30) Foreign Application Priority Data
Sep. 7, 2023 (JP) 2023-145533

(51) Int. Cl. C12M 1/00; C12N 1/066

(52) U.S. Cl. CPC C12M 29/04; C12M 47/06; C12N 1/066

(57) ABSTRACT

A filter that includes: a filter base body that has a first principal surface having a projection and a second principal surface on an opposite side from the first principal surface and that defines a plurality of through-holes through which the first principal surface communicates with the second principal surface. The projection is located between adjacent through-holes and projects in a first direction away from the filter base body. In the cross-section cut in a second direction in which the through-holes are adjacent to each other, the projection is located inward of an end portion of the filter base body located between the adjacent through-holes. The thickness of the projection in the second direction decreases toward the top portion of the projection.

### CROSS REFERENCE TO RELATED APPLICATIONS

The present application is a continuation of International application No. PCT/JP2024/031566, filed September 3, 2024, which claims priority to Japanese Patent Application No. 2023-145533, filed September 7, 2023, the entire contents of each of which are incorporated herein by reference.

### TECHNICAL FIELD

The present disclosure relates to a filter, a cell membrane disruption device, and a cell membrane disruption method.

### BACKGROUND ART

For example, Patent Document 1 discloses an intracellular substance extraction method including a step of suspending cells in a solution prepared by using benzalkonium chloride as a surfactant at a concentration in the range of 0.0005% to 0.01%, and a step of mixing a liquid having an osmotic pressure different from the intracellular osmotic pressure.

Patent Document 2 discloses a method in which, while a high-resistance (gigaohm) electrical seal is formed between a region of the cell membrane of a cell attached to a patch-clamp electrode and a tip opening of the electrode, the cell membrane at the sealed portion is disrupted. The method described in Patent Document 1 includes (a) a step of bringing a test cell into contact with a tip opening of a patch-clamp pipette, and (b) a step of applying negative pressure to the cell through the patch-clamp pipette, the negative pressure being controlled by a pressure calibrator.

Patent Document 1: Unexamined Patent Application Publication No. 2009-42

Patent Document 2: Unexamined Patent Application Publication No. 2004-294211

### SUMMARY OF THE DISCLOSURE

There has recently been a need for efficient disruption of cell membranes.

The present disclosure is directed to a filter, a cell membrane disruption device, and a cell membrane disruption method, each capable of efficiently disrupting cell membranes.

A filter according to one aspect of the present disclosure is a filter for disrupting cell membranes, the filter including: a filter base body that has a first principal surface having a projection and a second principal surface on an opposite side from the first principal surface and defines a plurality of through-holes through which the first principal surface communicates with the second principal surface, wherein the projection is located between through-holes adjacent to each other and projects in a first direction away from the filter base body, in a cross-section cut in a second direction in which the through-holes are adjacent to each other, the projection is located inward of an end portion of the filter base body located between the through-holes adjacent to each other, and a thickness of the projection in the second direction decreases toward a top portion of the projection.

A cell membrane disruption device according to one aspect of the present disclosure includes the filter according to the above aspect.

A cell membrane disruption method according to one aspect of the present disclosure includes: preparing a filter including a filter base body that has a first principal surface having a projection and a second principal surface on an opposite side from the first principal surface and that defines a plurality of through-holes through which the first principal surface communicates with the second principal surface; and introducing cells containing cell membranes onto a first principal surface side of the filter base body of the filter.

According to the present disclosure, a filter, a cell membrane disruption device, and a cell membrane disruption method, each capable of efficiently disrupting cell membranes, can be provided.

### BRIEF DESCRIPTION OF DRAWINGS

FIG. 1 is a schematic plan view of an example of a filter according to a first embodiment of the present disclosure as viewed from the first principal surface side.

FIG. 2 is an enlarged plan view of a portion of a filter part.

FIG. 3 is an enlarged perspective view of a portion of the filter part.

FIG. 4 is a schematic cross-sectional view of a portion of the filter part.

FIG. 5A is a schematic view illustrating an example of a step of manufacturing a filter.

FIG. 5B is a schematic view illustrating an example of a step of manufacturing the filter.

FIG. 5C is a schematic view illustrating an example of a step of manufacturing the filter.

FIG. 5D is a schematic view illustrating an example of a step of manufacturing the filter.

FIG. 5E is a schematic view illustrating an example of a step of manufacturing the filter.

FIG. 5F is a schematic view illustrating an example of a step of manufacturing the filter.

FIG. 5G is a schematic view illustrating an example of a step of manufacturing the filter.

FIG. 6 is a schematic view of an example of a cell membrane disruption device according to the first embodiment of the present disclosure.

FIG. 7 is a flowchart of an example of a cell membrane disruption method according to the first embodiment of the present disclosure.

FIG. 8A is a schematic view illustrating an example of a step in the cell membrane disruption method according to the first embodiment of the present disclosure.

FIG. 8B is a schematic view illustrating an example of a step in the cell membrane disruption method according to the first embodiment of the present disclosure.

FIG. 8C is a schematic view illustrating an example of a step in the cell membrane disruption method according to the first embodiment of the present disclosure.

FIG. 9 is a schematic view of an example of a cell membrane disruption device according to a second embodiment of the present disclosure.

FIG. 10 is a flowchart of an example of a cell membrane disruption method according to the second embodiment of the present disclosure.

FIG. 11 is a schematic cross-sectional view of a portion of a filter of a first modification.

FIG. 12 is a schematic cross-sectional view of a portion of a filter of a second modification.

FIG. 13 is a flowchart of an example of a cell membrane disruption method according to a third modification.

FIG. 14 is a schematic cross-sectional view of a portion of a filter of a fourth modification.

### DESCRIPTION OF THE PREFERRED EMBODIMENTS

The first embodiment according to the present disclosure will be described below with reference to the accompanying drawings. In the drawings, elements are exaggerated for ease of description.

### First Embodiment

### Filter

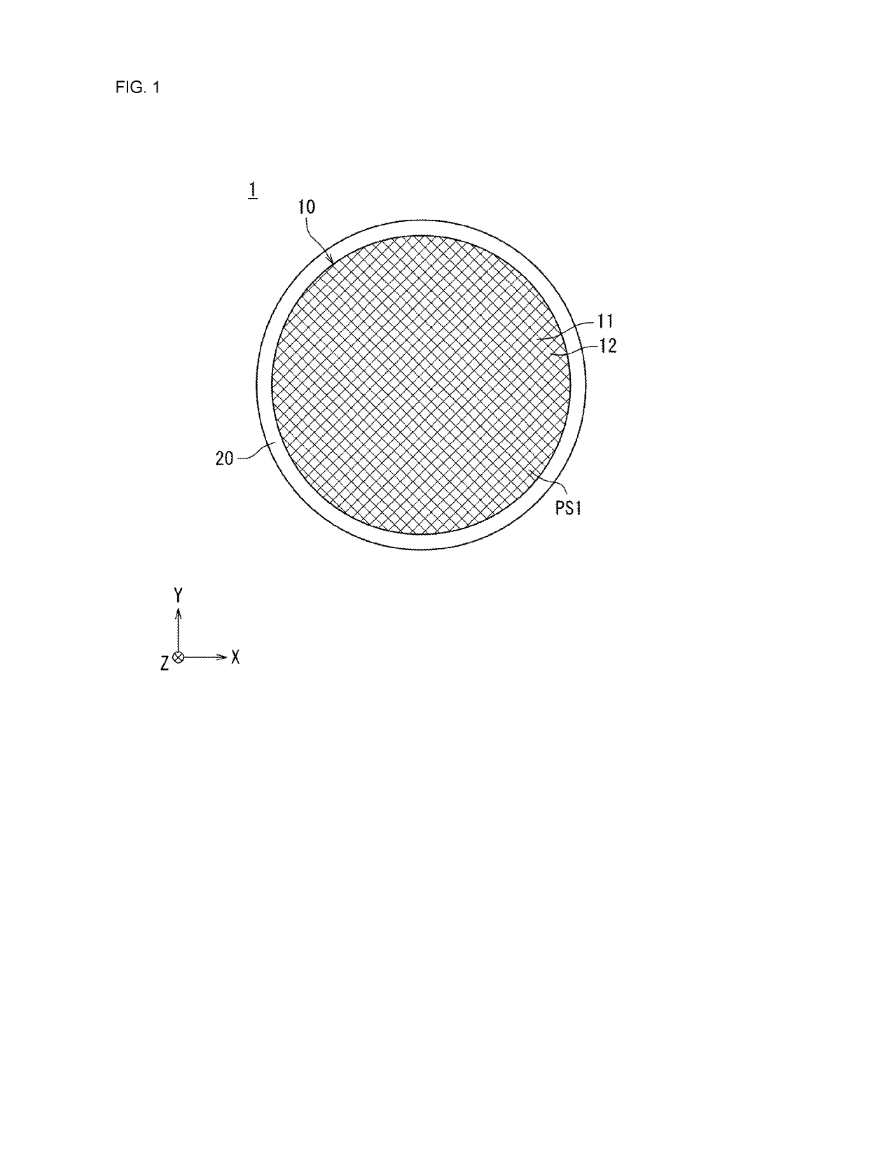

FIG. 1 is a schematic plan view of an example of a filter 1 according to a first embodiment of the present disclosure as viewed from the first principal surface PS1 side. In the figure, the X, Y, and Z directions indicate the vertical direction, horizontal direction, and thickness direction of the filter 1, respectively.

Referring to FIG. 1, the filter 1 disrupts cell membranes by filtering cells containing cell membranes. The filter 1 extracts cellular contents by disrupting the cell membranes.

The term "cells" refers to cells having cell membranes, such as induced pluripotent stem cells (iPS cells), ES cells, stem cells, mesenchymal stem cells, mononuclear cells, single cells, cell clusters, floating cells, adherent cells, nerve cells, leukocytes, cells for regenerative medicine, autologous cells, cancer cells, circulating tumor cells (CTCs), HL-60, HELA, eubacteria, fungi, archaea, and yeasts. Alternatively, the term "cell" includes substances having membranes containing lipids and proteins.

The term "cellular contents" refers to substances present inside a cell membrane, such as cytosol, nuclei, mitochondria, plastids, endoplasmic reticulum, Golgi body, lysosomes, peroxisomes, endosomes, genetic factors including RNA, and proteins.

The material of the filter 1 contains, as a main component, for example, at least one selected from metals, oxides, polymers, and ceramics. For metals and metal oxides, the material of the filter 1 may be, for example, gold, silver, copper, platinum, nickel, palladium, titanium, aluminum, an alloy thereof, or an oxide thereof. For polymers, the material of the filter 1 may be, for example, polyethylene or polypropylene. In particular, the use of a nickel-palladium alloy, titanium, aluminum, an oxide thereof, or polyethylene can reduce eluted components and can minimize effects on cells and cellular contents. In addition, a small amount of eluted components can reduce noise in the recovery or analysis of the cellular contents.

The filter 1 includes a filter part 10 and a frame part 20 at the outer periphery of the filter part 10. The filter part 10 has a plurality of through-holes 11 and disrupts cell membranes to extract cellular contents. The frame part 20 is provided at the outer periphery of the filter part 10 and is used to attach the filter 1 to a container or a device. Alternatively, the frame part 20 is grasped by forceps.

In the first embodiment, the filter part 10 and the frame part 20 are formed integrally.

### Filter Part

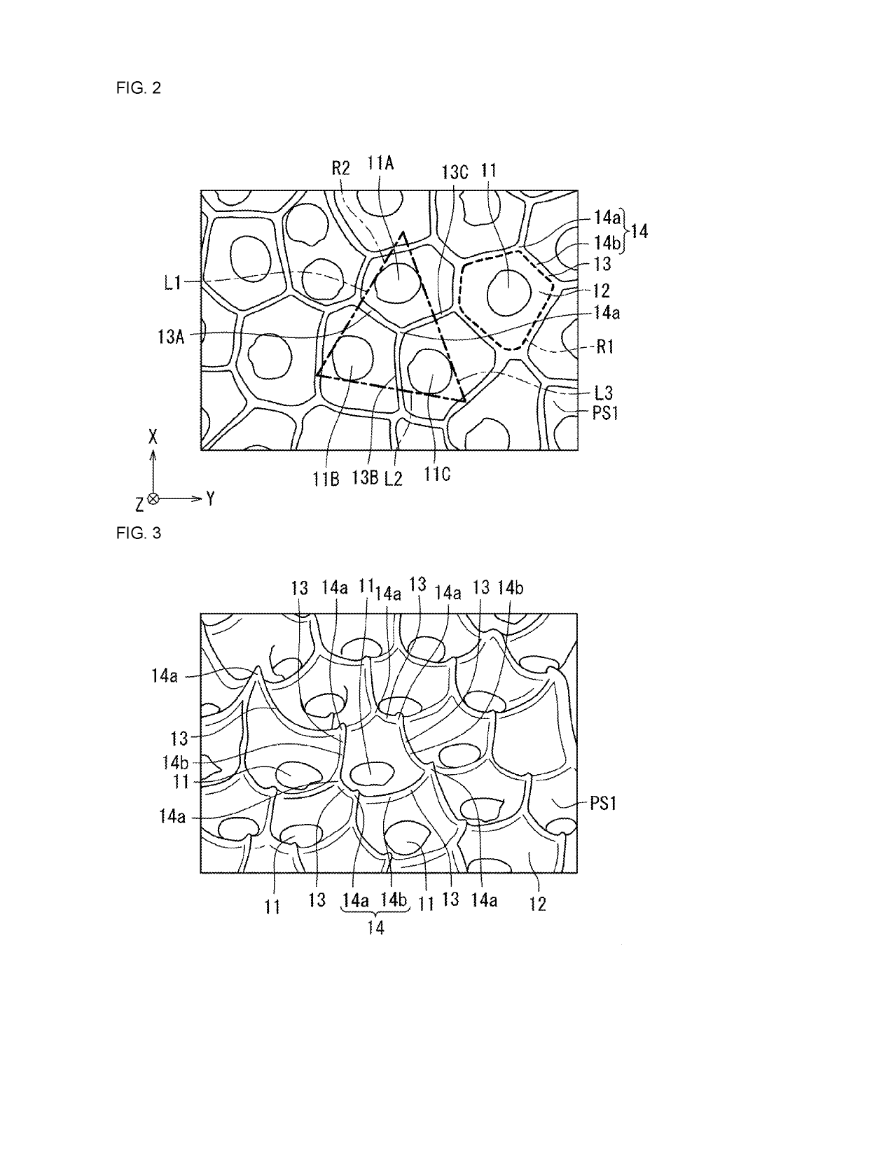

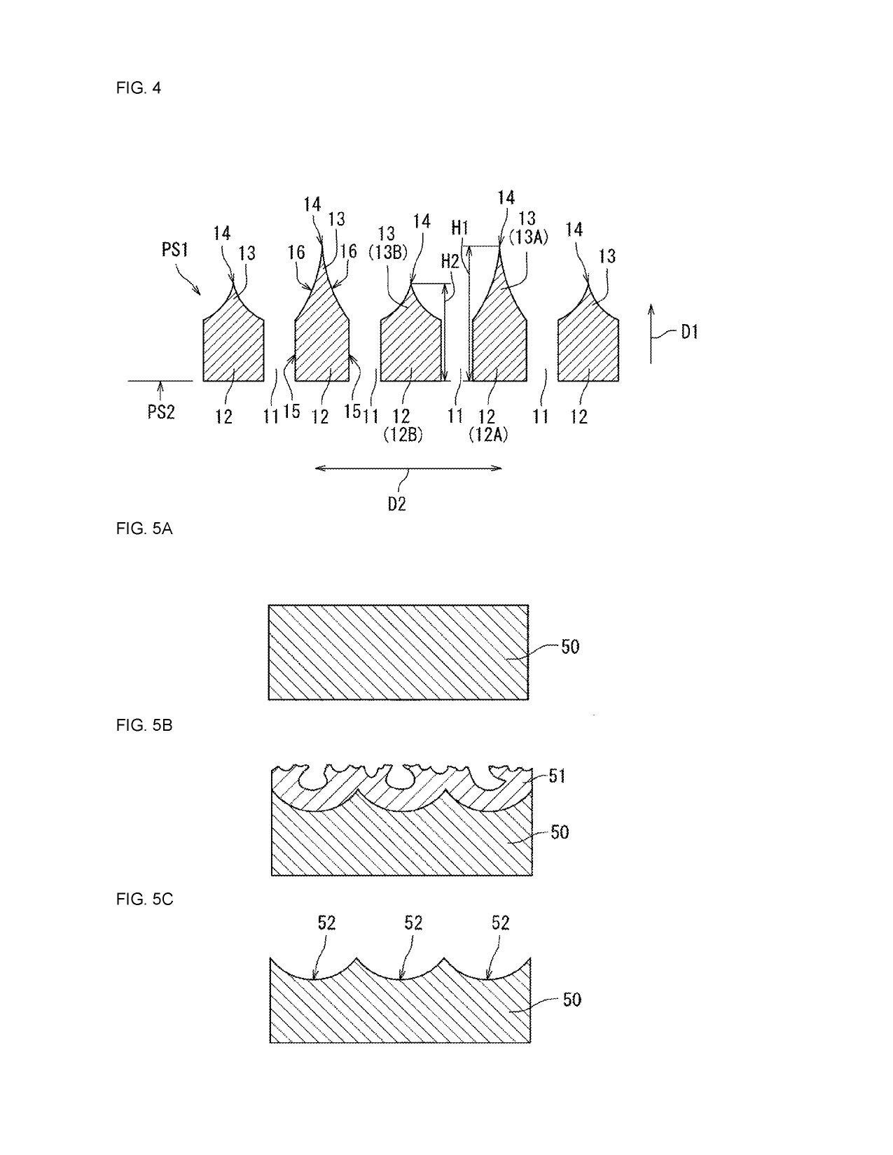

FIG. 2 is an enlarged plan view of a portion of the filter part 10. FIG. 3 is an enlarged perspective view of a portion of the filter part 10. FIG. 4 is a schematic cross-sectional view of a portion of the filter part 10.

Referring to FIGS. 2 to 4, the filter part 10 includes a filter base body 12 that defines the plurality of through-holes 11. The filter base body 12 has a projection 13.

The filter base body 12 has a first principal surface PS1 having the projection 13 and a second principal surface PS2 on the opposite side from the first principal surface PS1. For example, the first principal surface PS1 includes an uneven surface, and the uneven surface is formed by the projection 13. The second principal surface PS2 includes a flat surface. The filter base body 12 defines the plurality of through-holes 11 through which the first principal surface PS1 communicates with the second principal surface PS2.

The plurality of through-holes 11 have a substantially circular shape as viewed from the first principal surface PS1 side of the filter base body 12, namely in the Z direction. The term "substantially circular shape" includes a true circular shape and an elliptical shape where the difference in length between the major axis and the minor axis is within 10%.

For example, the diameter of the through-holes 11 is appropriately designed according to the type (size, morphology, properties, elasticity) or amount of cells to be disrupted. The diameter of the through-holes 11 is, for example, 5 nm to 200 μm. This makes it possible to process target cells ranging from small fungi having a size of about 5 nm to cell clusters (aggregates of cells) having an overall size of about 200 μm. The diameter of the through-holes 11 is preferably 5 nm to 40 μm. This allows efficient processing of isolated cells. The diameter is more preferably 5 nm to 5 μm. This facilitates the passage of cellular contents, such as cell nuclei, through the through-holes 11 and prevents intact cells from passing through the through-holes 11, enabling efficient collection of cellular contents from the cells. Preferably, the through-holes 11 penetrate, but do not branch, between the two principal surfaces. This configuration prevents cellular contents passing through the through-holes 11 from being trapped within the through-holes 11 and thus increases the recovery rate of cellular contents. The diameter of each through-hole 11 can be measured using an SEM and an image analysis application (Hakuto Co., Ltd., product name: Image-Pro (registered trademark)). For example, an SEM image of holes is analyzed using the image analysis application (Hakuto Co., Ltd., product name: Image-Pro (registered trademark)) to identify the contour of each hole. The contour of each hole may be automatically identified by the image analysis application or may be manually identified by a user operating an input interface. Next, the centroid of a planar shape defined by the contour of each hole is calculated, and a long side and a short side of the planar shape are calculated based on the centroid. The average value of the long side and the short side of the planar shape is calculated as the diameter of the through-hole 11.

For example, the spacing between adjacent through-holes 11 is greater than 1 time the diameter of the through-holes 11 and less than or equal to 10 times the diameter of the through-holes 11. Preferably, the spacing between adjacent through-holes 11 is less than or equal to 3 times the diameter of the through-holes 11. For example, the spacing between adjacent through-holes 11 is 10 nm to 600 μm. More preferably, the spacing between adjacent through-holes 11 is 10 nm to 120 μm. Still more preferably, the spacing between adjacent through-holes 11 is 10 nm to 15 μm. The disruption area ratio can be controlled by adjusting the spacing between adjacent through-holes 11.

The shapes of the plurality of through-holes 11 are not limited to substantially circular shapes as viewed from the first principal surface PS1 side. For example, the shapes of the plurality of through-holes 11 may include triangular shapes, rectangular shapes, or polygonal shapes as viewed from the first principal surface PS1 side. The sizes and shapes of the plurality of through-holes 11 are appropriately designed according to the size, morphology, properties, elasticity, or amount of cells or intracellular substances.

The sizes and spacing of the plurality of through-holes 11 may be different or may be the same.

For example, the opening ratio of the filter part 10 is 10% or more, and the opening ratio is preferably 25% or more. The opening ratio is calculated as (the area occupied by the through-holes 11)/(the projected area of the first principal surface PS1 assuming that no through-holes 11 are open).

The filter base body 12 has the projection 13 on the first principal surface PS1 side. The projection 13 is located between through-holes 11 adjacent to each other. The projection 13 projects in a first direction D1 away from the filter base body 12. The "first direction D1" may be parallel to the thickness direction (Z direction) of the filter 1 or may be inclined with respect to the thickness direction.

The thickness of the projection 13 decreases toward a top portion 14 of the projection 13. In other words, the thickness of the projection 13 decreases with distance from the filter base body 12 in the first direction D1. The thickness of the projection 13 is a dimension of the projection 13 in a second direction D2 in which the through-holes 11 are adjacent to each other.

As viewed from the first principal surface PS1 side of the filter base body 12, the projection 13 surrounds one or more through-holes 11. For example, the projection 13 has a cylindrical shape surrounding one or more through-holes 11. For example, the projection 13 surrounds one to three through-holes 11. Preferably, the projection 13 surrounds one through-hole 11.

Referring to FIG. 2, as viewed from the first principal surface side of the filter base body 12, the top portion 14 of the projection 13 is curved and surrounds one or more through-holes 11. The top portion 14 of the projection 13 is a distal end of the projection 13 in the first direction D1. As viewed from the first principal surface side of the filter base body 12, the area of a region R1 surrounded by the top portion 14 of the projection 13 is larger than the opening area of one or more through-holes 11. For example, the area of the region R1 is greater than 1 time and less than or equal to 36/π times the opening area of one or more through-holes 11. The top portion 14 being curved allows the area of cell membrane disruption to be larger than the area of the projection 13 and can thus delay cell membrane repair.

Referring to FIG. 4, in the cross-section of the filter 1 cut in the second direction D2, the projection 13 is located inward of an end portion 15 of the filter base body 12 in the second direction D2, which is located between through-holes 11 adjacent to each other. The projection 13 is located inward of the end portion 15 of the filter base body 12 in the second direction D2 and projects in the first direction D1. Specifically, the top portion 14 of the projection 13 is located inward of the end portion 15 of the filter base body 12 in the second direction D2.

The projection 13 has an outer surface 16 that curves toward the end portion 15 from a central region of the filter base body 12 in the second direction D2, which is located between through-holes 11 adjacent to each other. The central region refers to a region within 40% of a dimension of the filter base body 12 in the second direction D2 from a center of the filter base body 12 in the second direction D2. For example, the outer surface 16 is curved in a rounded manner. In the cross-section of the filter 1 cut in the second direction D2, the radius of curvature of the outer surface 16 is appropriately designed according to the thickness of the filter 1 (the distance between the first principal surface PS1 and the second principal surface PS2) or the diameter of the through-holes 11. For example, the radius of curvature of the outer surface 16 is 1 nm to 200 μm.

Referring to FIGS. 3 and 4, the height of the projection 13 varies. For example, the height of the projection 13 continuously changes. The height of the projection 13 refers to the distance from the second principal surface PS2 of the filter base body 12 to the top portion 14 of the projection 13. For example, the height of the projection 13 is a distance from the top portion 14 of the projection 13 to the second principal surface PS2 in a direction perpendicular to the second principal surface PS2 of the filter base body 12.

For example, the height of the projection 13 is 5 nm to 100 μm such that the projection 13 can disrupt the cell membrane and puncture from the cell membrane into the interior of the cell. To thin the filter 1 and reduce the pressure loss of a fluid passing through the filter 1, the height of the projection 13 is preferably 5 nm to 1 μm. The diameter ratio of the projection 13 to the through-holes 11 is from 1:1 to 1:0.2.

Referring to FIG. 4, attention is directed to a first projection 13A on a first filter base body 12A and a second projection 13B on a second filter base body 12B.

The first filter base body 12A and the second filter base body 12B form part of the filter base body 12. The first filter base body 12A is located between through-holes 11 adjacent to each other. The second filter base body 12B is different from the first filter base body 12A and is located between through-holes 11 adjacent to each other.

In the example illustrated in FIG. 4, the first filter base body 12A is adjacent to the second filter base body 12B. The first filter base body 12A is not necessarily adjacent to the second filter base body 12B. The first filter base body 12A and the second filter base body 12B are provided at different positions in the filter base body 12.

The first projection 13A and the second projection 13B form part of the projection 13. The first projection 13A projects from the first principal surface PS1 of the first filter base body 12A. The second projection 13B projects from the first principal surface PS1 of the second filter base body 12B.

The height H1 of the first projection 13A differs from the height H2 of the second projection 13B. In the example illustrated in FIG. 4, the height H1 of the first projection 13A is greater than the height H2 of the second projection 13B.

Referring to FIG. 3, the projection 13 has pointed top portions 14a. The pointed top portions 14a each have a shape in which the distal end of the projection 13 is tapered. The pointed top portions 14a are provided in at least part of the top portion 14 of the projection 13. In the top portion 14 of the projection 13, different top portions 14b other than the pointed top portions 14a may have either a pointed shape or a non-pointed shape. The different top portions 14b may be rounded.

As viewed from the first principal surface PS1 side of the filter base body 12, a region R2 is drawn by connecting three virtual straight lines L1, L2, and L3 each being in contact with the outer peripheries of two adjacent through-holes among three through-holes 11A, 11B, and 11C adjacent to each other such that the three through-holes 11A, 11B, and 11C are included in the region R2. The pointed top portion 14a is located within the region R2.

For example, as illustrated in FIG. 2, as viewed from the first principal surface PS1 side of the filter base body 12, the first virtual straight line L1, the second virtual straight line L2, and the third virtual straight line L3 each being in contact with the outer peripheries of the first through-hole 11A, the second through-hole 11B, and the third through-hole 11C adjacent to each other are drawn.

The first virtual straight line L1 is in contact with the outer periphery of the first through-hole 11A and the outer periphery of the second through-hole 11B. The second virtual straight line L2 is in contact with the outer periphery of the second through-hole 11B and the outer periphery of the third through-hole 11C. The third virtual straight line L3 is in contact with the outer periphery of the first through-hole 11A and the outer periphery of the third through-hole 11C.

As viewed from the first principal surface PS1 side of the filter base body 12, the region R2 is surrounded by the first virtual straight line L1, the second virtual straight line L2, and the third virtual straight line L3, and has a triangular shape. The first through-hole 11A, the second through-hole 11B, and the third through-hole 11C are located within the region R2.

For example, as viewed from the first principal surface PS1 side of the filter base body 12, the pointed top portion 14a is located at a central position of the region R2.

The height of the projection 13 at the pointed top portion 14a is greater than the height of the projection 13 at the different top portions 14b. For example, the height of the projection 13 at the pointed top portion 14a is greater than 1 time and equal to or less than 2 times the height of the projection 13 at the different top portions 14b.

### Frame Part

Referring back to FIG. 1, the frame part 20 is provided at the outer periphery of the filter part 10 and has a smaller number of through-holes 11 per unit area than the filter part 10. The number of through-holes 11 in the frame part 20 is 25% or less of the number of through-holes 11 in the filter part 10.

The frame part 20 has a ring shape as viewed from the first principal surface PS1 side of the filter part 10. As the filter 1 is viewed from the first principal surface PS1 side, the center of the frame part 20 coincides with the center of the filter part 10. In other words, the frame part 20 is concentric with the filter 1.

### Filter Manufacturing

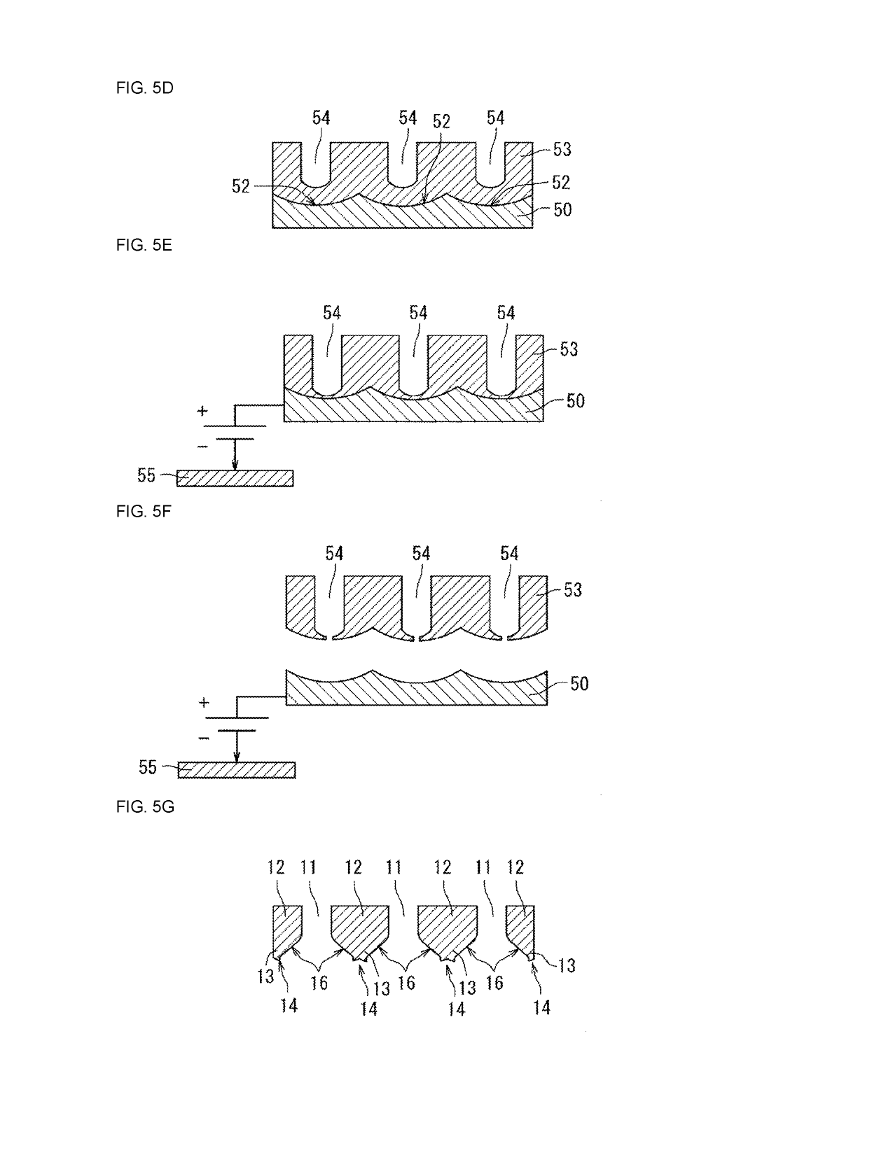

An example method for manufacturing the filter 1 will be described with reference to FIGS. 5A to 5G. FIGS. 5A to 5G are schematic views illustrating examples of the steps of manufacturing the filter 1.

Referring to FIG. 5A, an aluminum plate 50 is subjected to electropolishing. This process flattens the surface of the aluminum plate 50.

Referring to FIG. 5B, the aluminum plate 50 is subjected to anodic oxidation treatment. For example, the aluminum plate 50 is subjected to anodic electrolytic treatment. This treatment forms a first oxide film 51 on the surface layer of the aluminum plate 50. For example, the first oxide film 51 has an irregular outer surface.

Referring to FIG. 5C, the first oxide film 51 is removed from the aluminum plate 50. For example, the first oxide film 51 is etched away from the aluminum plate 50. This process forms a plurality of recesses 52 in regions of the aluminum plate 50 from which the first oxide film 51 has been removed. For example, the recesses 52 each have a rounded shape.

Referring to FIG. 5D, the aluminum plate 50 having the recesses 52 is subjected to anodic oxidation treatment. This treatment forms a second oxide film 53 on a surface of the aluminum plate 50 that has the recesses 52 thereon. The second oxide film 53 has a plurality of holes 54 extending toward the bottoms of the recesses 52.

Referring to FIGS. 5E and 5F, the aluminum plate 50 is peeled off from the second oxide film 53. For example, the aluminum plate 50 is peeled off from the second oxide film 53 by anodic electrolytic stripping using the aluminum plate 50 as an anode and a cathode plate 55 as a cathode.

Alternatively, the aluminum plate 50 may be peeled off by cathodic electrolytic stripping using the aluminum plate 50 as a cathode.

Referring to FIG. 5G, the bottom of the second oxide film 53 is etched to open the plurality of holes 54 and form the filter base body 12 that defines the plurality of through-holes 11. The through-holes 11 with a desired hole size are formed by performing an etching process so as to enlarge the plurality of holes 54.

The filter 1 can be produced accordingly.

### Cell Membrane Disruption Device

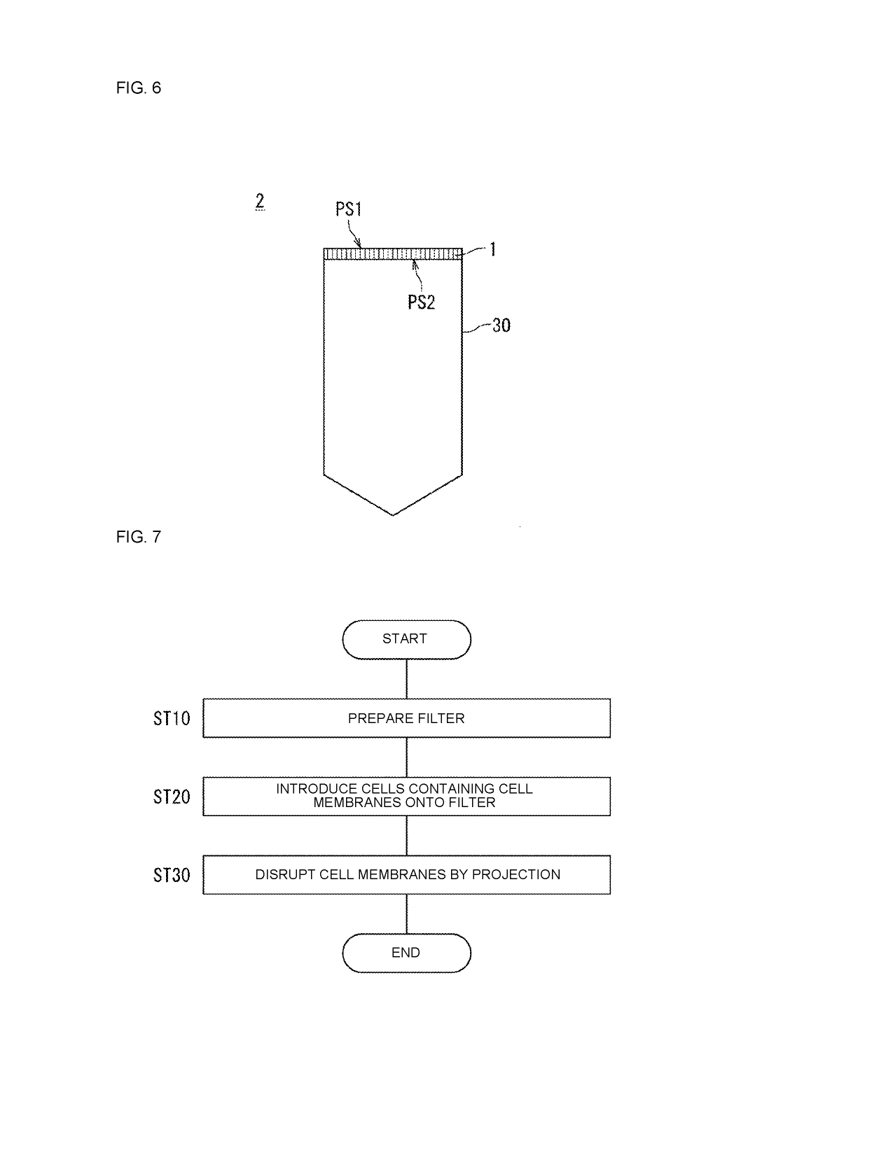

The cell membrane disruption device will be described with reference to FIG. 6. FIG. 6 is a schematic diagram of an example of a cell membrane disruption device 2 according to the first embodiment of the present disclosure.

Referring to FIG. 6, the cell membrane disruption device 2 includes the filter 1 and a container 30 to which the filter 1 is attached.

The container 30 has an opening. The filter 1 is attached to the opening of the container 30. For example, the container 30 may be a centrifuge tube, a test tube, a microtube, a flask, or a Petri dish.

### Cell Membrane Disruption Method

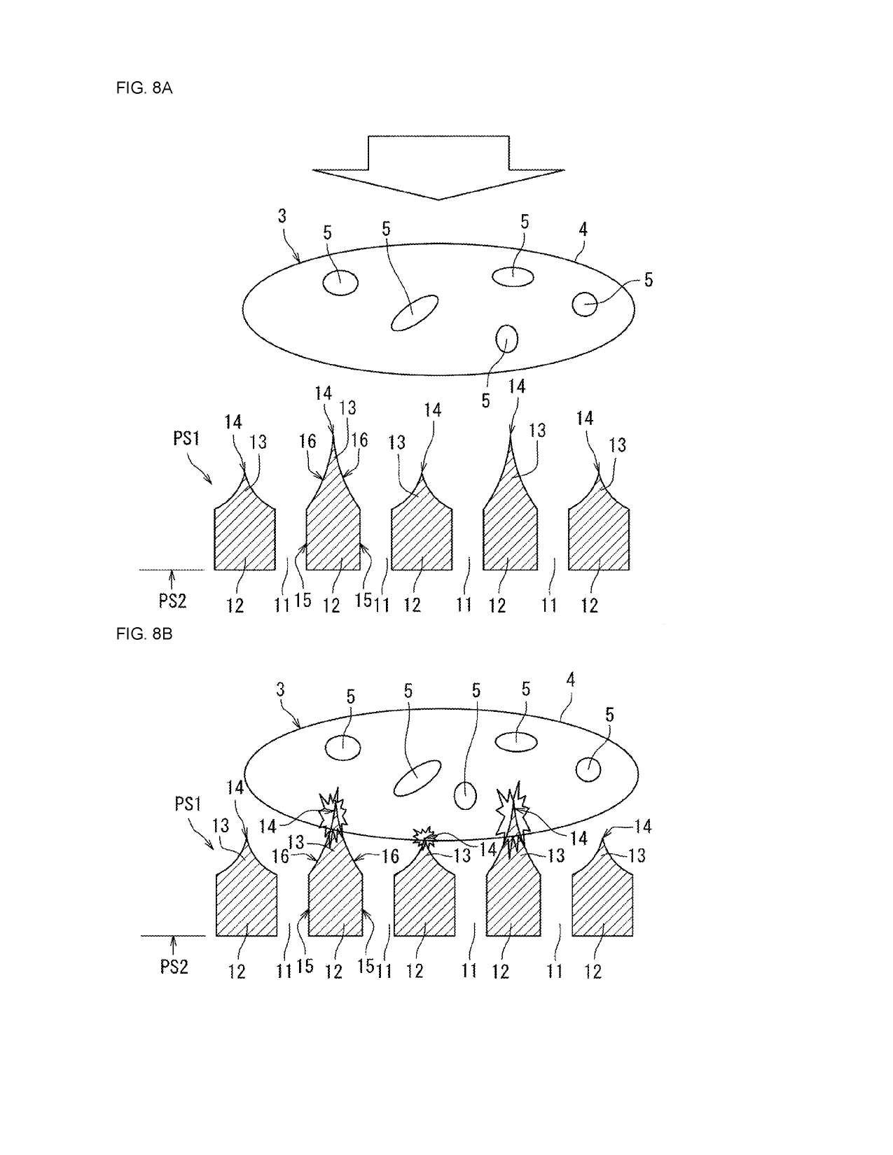

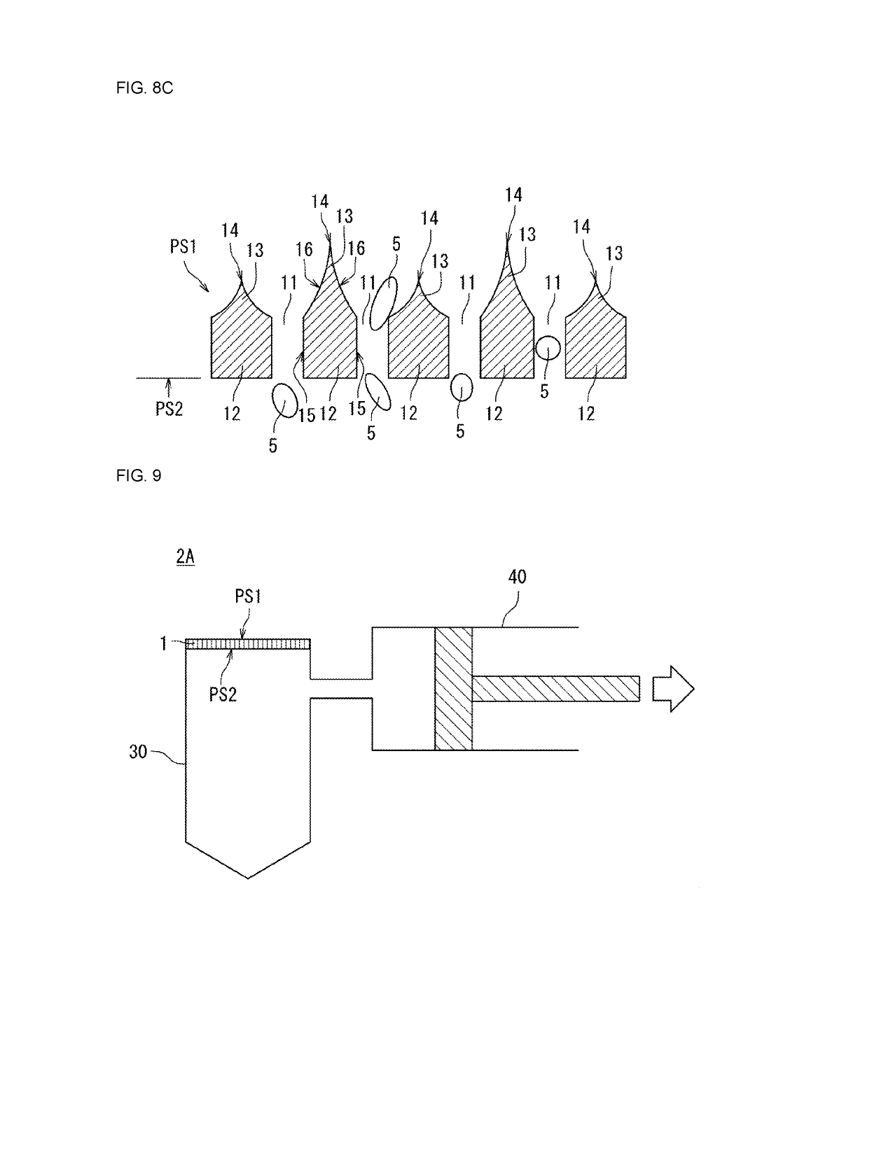

The cell membrane disruption method will be described with reference to FIGS. 7 and 8A to 8C. FIG. 7 is a flowchart of an example of the cell membrane disruption method according to the first embodiment of the present disclosure. FIGS. 8A to 8C are schematic diagrams illustrating examples of steps in the cell membrane disruption method according to the first embodiment of the present disclosure.

Referring to FIG. 7, the filter 1 is prepared in a step ST10.

In a step ST20, cells containing cell membranes are introduced onto the filter 1. For example, a cell suspension containing a plurality of cells is introduced onto the first principal surface PS1 side of the filter base body 12 of the filter 1.

Referring to FIG. 8A, a cell 3 has a plurality of cellular contents 5 in a cell membrane 4. In the step ST20, the cell 3 having the cell membrane 4 is introduced onto the first principal surface PS1 side of the filter base body 12 of the filter 1.

Referring back to FIG. 7, in a step ST30, the cell membrane 4 is disrupted by the projection 13 of the filter 1. Referring to FIG. 8B, the cell 3 that has been introduced onto the first principal surface PS1 side of the filter base body 12 falls vertically downward due to gravity. The first principal surface PS1 of the filter base body 12 has the projection 13. The cell membrane 4 of the cell 3 is pierced by the top portion 14 of the projection 13 and is disrupted. This causes the cellular contents 5 to be discharged outside the cell membrane 4.

Referring to FIG. 8C, the plurality of through-holes 11 in the filter 1 have a size that allows the cellular contents 5 to pass through. Consequently, the cellular contents 5 discharged outside the cell membrane 4 pass through the through-holes 11. The cell membrane 4 is captured by the filter 1.

Accordingly, the cell membrane 4 of the cell 3 can be disrupted using the filter 1.

### Advantageous Effects

The filter 1 according to the first embodiment can provide the following advantageous effects.

The filter 1 disrupts the cell membrane 4 and includes the filter base body 12. The filter base body 12 has the first principal surface PS1 having the projection 13 and the second principal surface PS2 on the opposite side from the first principal surface PS1 and defines the plurality of through-holes 11 through which the first principal surface PS1 communicates with the second principal surface PS2. The projection 13 is located between through-holes 11 adjacent to each other and projects in the first direction D1 away from the filter base body 12. In the cross-section cut in the second direction D2 in which the through-holes 11 are adjacent to each other, the projection 13 is located inward of the end portion 15 of the filter base body 12 located between the through-holes 11 adjacent to each other. The thickness of the projection 13 in the second direction D2 decreases toward the top portion 14 of the projection 13. This configuration makes the periphery of the projection 13 substantially equal to the periphery of the through-holes 11 and improves the disruption rate.

This configuration enables efficient disruption of the cell membrane 4. According to the filter 1, the projection 13 of the filter 1 can disrupt the cell membrane 4. This allows the cell membrane 4 to be disrupted in a relatively short time. Furthermore, the projection 13 can puncture and disrupt a plurality of sites of a single cell substantially simultaneously. This allows the cell membrane to be disrupted in an even shorter time and enables the extraction of cellular contents from a plurality of sites. Additionally, a relatively large number of cells 3 can be processed at once. Furthermore, the recovery of the cellular contents through the through-holes 11 after disrupting the cell membrane 4 eliminates the need for post-disruption processing or simplifies such processing. Furthermore, the disruption of the majority of the introduced cells in a short time can reduce clogging of the filter 1 and can reduce the pressure loss of a fluid passing through the filter 1. Moreover, the reduced pressure loss and the like can reduce heat generation, thereby reducing the inactivation of the cellular contents (e.g., cellular contents containing proteins).

In the present disclosure, efficient disruption of the cell membrane 4 may be determined based on the following factors.

(a) Disruption rate. The number of cells disrupted by the filter relative to the total number of introduced cells.

(b) Disruption speed. The number of cells disrupted by the filter per unit time.

(c) Disruption area ratio. The ratio of the area of voids opened per cell by the filter to the surface area of the cell.

(d) Disruption yield rate. The ratio of the number of cells disrupted by the filter to the activity of biological substances obtained by the disruption. General means of expressing the activity of biological substances include mass, concentration, potential, and fluorescence spectrum.

(e) Disruptable range. The range of diameters of cells that can be disrupted by the filter.

In the second direction D2, the projection 13 has the outer surface 16 that curves toward the end portion 15 from a central region of the filter base body 12 located between through-holes 11 adjacent to each other. This configuration facilitates disruption of the cell membrane 4. This configuration also allows the cellular contents 5, which have been discharged outside the cell membrane 4, to flow more readily into the through-holes 11, thereby reducing the pressure loss of the fluid passing through the filter 1.

The projection 13 has a flat inclined surface 16a, which is inclined toward the through-holes 11, in at least part of the outer surface 16 of the projection 13. This configuration facilitates the flow of the cellular contents 5, which have been discharged outside the cell membrane 4, into the through-holes 11.

The filter base body 12 includes the first filter base body 12A, which is located between through-holes 11 adjacent to each other, and the second filter base body 12B, which is different from the first filter base body 12A and is located between through-holes 11 adjacent to each other. The projection 13 includes the first projection 13A, which projects from the first principal surface PS1 of the first filter base body 12A, and the second projection 13B, which projects from the second principal surface PS2 of the second filter base body 12B. When the height of the projection 13 is defined as the distance from the second principal surface PS2 of the filter base body 12 to the top portion 14 of the projection 13, the height H1 of the first projection 13A differs from the height H2 of the second projection 13B. This configuration enables more efficient disruption of the cell membrane 4. For example, when the cells 3 introduced onto the filter 1 vary in size, larger cells tend to be disrupted by the projection 13 having a greater height, and smaller cells tend to be disrupted by the projection 13 having a smaller height. This can increase the surface area of the filter 1 used for disruption and can shorten the processing time to accelerate the disruption speed. This configuration also allows more cells to be processed at once. Furthermore, it is possible to expand the disruptable range and disrupt the cells 3 having the cell membranes 4 of various types and sizes.

As viewed from the first principal surface PS1 side of the filter base body 12, the projection 13 surrounds one or more through-holes 11. This configuration allows the cell membrane to be disrupted before the cell reaches the through-holes 11 and thus enables more efficient disruption of the cell membrane 4.

The height of the projection 13 continuously changes. This configuration facilitates disruption of the cell membrane 4 and enables more efficient disruption of the cell membrane 4. After the cell membrane is punctured by the top portion 14 of the projection 13, the holes formed in the cell membrane can be gradually enlarged. This allows disruption of the cell membrane with minimal force and facilitates extraction of the cellular contents. Additionally, the pressure loss of the fluid passing through the filter 1 can be reduced.

As viewed from the first principal surface PS1 side of the filter base body 12, the area of a region R1 surrounded by the top portion 14 of the projection 13 is larger than the opening area of one or more through-holes 11. This configuration enables more efficient disruption of the cell membrane 4. According to this configuration, the projection 13 does not block the through-holes 11 as viewed from the first principal surface PS1 side, resulting in no pressure loss in the filter.

The projection 13 has the pointed top portions 14a. This configuration facilitates disruption of the cell membrane 4 and enables more efficient disruption of the cell membrane 4.

When the region R2 is drawn by connecting three virtual straight lines L1, L2, and L3 each being in contact with the outer peripheries of two adjacent through-holes among three through-holes 11A, 11B, and 11C adjacent to each other such that the three through-holes 11A, 11B, and 11C are included in the region R2 as viewed from the first principal surface PS1 side of the filter base body 12, the pointed top portion 14a is located within the region R2. This configuration facilitates disruption of the cell membrane 4 and enables more efficient disruption of the cell membrane 4. Since the pointed top portion 14a is close to the through-holes 11A, 11B, 11C through which the cellular contents pass, the cellular contents can pass through the through-holes 11A, 11B, 11C more easily, thereby increasing the recovery rate of the cellular contents.

The height of the projection 13 at the pointed top portion 14a is greater than the height of the projection 13 at the different top portions 14b. This configuration facilitates disruption of the cell membrane 4 and enables more efficient disruption of the cell membrane 4. After the cell membrane 4 is punctured by the top portion 14 of the projection 13, the holes formed in the cell membrane can be gradually enlarged. This allows disruption of the cell membrane with minimal force and facilitates extraction of the cellular contents. Additionally, the pressure loss of the fluid passing through the filter 1 can be reduced.

The spacing between adjacent through-holes 11 in the filter 1 is greater than 1 time the diameter of the through-holes 11 and less than or equal to 10 times the diameter of the through-holes 11. Preferably, the spacing between adjacent through-holes 11 is less than or equal to 3 times the diameter of the through-holes 11. For example, the spacing between adjacent through-holes 11 is 10 nm to 600 μm. More preferably, the spacing between adjacent through-holes 11 is 10 nm to 120 μm. Still more preferably, the spacing between adjacent through-holes 11 is 10 nm to 15 μm. According to this configuration, the liquid permeability can be increased by narrowing the spacing between the through-holes 11 to increase the opening ratio.

As viewed from the first principal surface PS1 side of the filter base body 12, the projection 13 surrounds one or more through-holes 11. For example, the projection 13 has a cylindrical shape surrounding one or more through-holes 11. For example, the projection 13 surrounds one to three through-holes 11. Preferably, the projection 13 surrounds one through-hole 11. This configuration enhances the strength of the filter base body 12 in the tensile direction.

The disruption device 2 for the cell membranes 4 includes the filter 1 according to the above aspect. Therefore, the disruption device 2 also provides the same advantageous effects as the filter 1.

The method for disrupting the cell membranes 4 includes: the step ST10 of preparing the filter 1; the step ST20 of introducing the cells 3 containing the cell membranes 4 onto the first principal surface PS1 side of the filter base body 12 of the filter 1; and the step ST30 of disrupting the cell membranes 4 using the projection 13 of the filter 1. This configuration enables efficient disruption of the cell membranes 4. Therefore, the cell membranes 4 can be disrupted in a relatively short time. Furthermore, a relatively large number of cells can be processed at once. Additionally, this method eliminates the need for processing after disruption of the cell membranes 4 or simplifies such processing.

This embodiment describes an example where the filter 1 includes the frame part 20; however, the present disclosure is not limited to this embodiment. For example, the filter 1 does not necessarily include the frame part 20. The entire filter 1 may be composed only of the filter part 10.

This embodiment describes an example where the projection 13 is provided on the first principal surface PS1 of the filter base body 12; however, the present disclosure is not limited to this embodiment. For example, the projection 13 may be provided on the second principal surface PS2 of the filter base body 12.

This embodiment describes an example where only the projection 13 is used to disrupt the cell membranes 4; however, the top portions 14b formed by providing the projection 13 may also be used to disrupt the cell membranes 4. This configuration increases the diameter of the holes created by puncturing to extend the time required for repair of the cell membranes.

This embodiment describes an example where the plurality of through-holes 11 have a substantially circular shape and are arranged at different spacings; however, the present disclosure is not limited to this embodiment. For example, the plurality of through-holes 11 may have a square shape. In this case, the plurality of through-holes 11 may be arranged in two array directions parallel to the sides of the square as viewed from the first principal surface PS1 side (Z direction) of the filter 1. The arrangement of the plurality of through-holes 11 in a square grid pattern can increase the opening ratio and can improve the liquid permeability of the filter 1.

The arrangement of the plurality of through-holes 11 is not limited to a square grid pattern; the plurality of through-holes 11 may be arranged, for example, in a quasi-periodic pattern or a periodic pattern. Examples of the periodic pattern include a rectangular array where the spacings between the through-holes 11 in two array directions are not equal, as well as a triangular grid pattern and an equilateral triangular grid pattern.

### Second Embodiment

A cell membrane disruption device and a cell membrane disruption method according to a second embodiment of the present disclosure will be described below. In the second embodiment, components identical or equivalent to those in the first embodiment are assigned the same reference signs, and descriptions overlapping with those of the first embodiment are omitted.

FIG. 9 is a schematic diagram of an example of a disruption device 2A for the cell membranes 4 according to the second embodiment of the present disclosure.

In the second embodiment, the disruption device 2A differs from that in the first embodiment in that the disruption device 2A includes a suction device 40.

Referring to FIG. 9, the disruption device 2A includes the suction device 40.

The suction device 40 draws the cells 3 containing the cell membranes 4, which are located on the first principal surface PS1 side of the filter base body 12, through the plurality of through-holes 11 to the second principal surface PS2 side.

The suction device 40 is connected to the container 30 at a position downstream of the filter 1. The suction device 40 draws gas in the container 30. By drawing gas in the container 30, the suction device 40 draws the cells 3 through the plurality of through-holes 11 of the filter 1 in the direction from the first principal surface PS1 to the second principal surface PS2 of the filter base body 12. For example, the suction device 40 is a syringe.

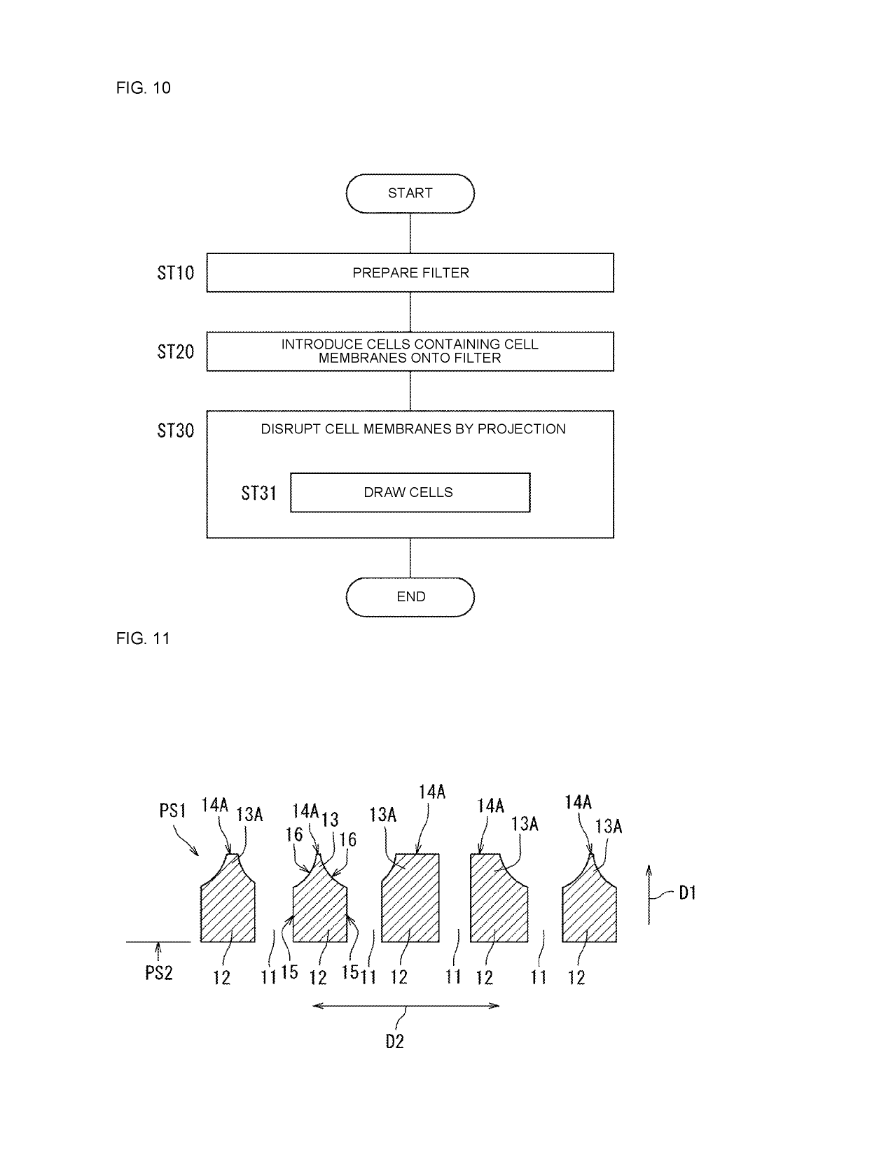

FIG. 10 is a flowchart of an example of the method for disrupting the cell membranes 4 according to the second embodiment of the present disclosure.

In this embodiment, the step ST30 of disrupting the cell membranes 4 includes a step ST31 of drawing the cells 3.

In the step ST31, the suction device 40 draws the cells 3, which are located on the first principal surface PS1 side of the filter base body 12, through the plurality of through-holes 11 to the second principal surface PS2 side.

In this embodiment, gas in the container 30 is drawn at a position downstream of the filter 1 by the suction device 40. Accordingly, the cells 3 are drawn in the direction from the first principal surface PS1 to the second principal surface PS2 of the filter base body 12.

### Advantageous Effects

The disruption device 2A for the cell membranes 4 according to the second embodiment can provide the following advantageous effects.

The disruption device 2A includes the suction device 40 that draws the cells 3 containing the cell membranes 4, which are located on the first principal surface PS1 side of the filter base body 12, through the plurality of through-holes 11 to the second principal surface PS2 side. This configuration facilitates disruption of the cell membranes 4. For example, when the cells 3 are drawn, the cell membranes 4 are pulled in the drawing direction and are more likely to be pierced by the projection 13. This enables more efficient disruption of the cell membranes 4.

In the disruption device 2A, gas in the container 30 to which the filter 1 is attached is drawn at a position downstream of the filter 1. This configuration enables more efficient disruption of the cell membranes 4.

In the method for disrupting the cell membranes 4, the step ST30 of disrupting the cell membranes 4 includes the step ST31 of drawing the cells 3, which are located on the first principal surface PS1 side of the filter base body 12, through the plurality of through-holes 11 to the second principal surface PS2 side. This configuration facilitates disruption of the cell membranes 4 and enables more efficient disruption of the cell membranes 4.

In the method for disrupting the cell membranes 4, the preparation step ST10 includes preparing the filter 1 attached to the container 30. The drawing step ST31 includes drawing the gas in the container 30 at a position downstream of the filter 1. This configuration enables more efficient disruption of the cell membranes 4.

This embodiment describes an example where the suction device 40 is a syringe; however, the present disclosure is not limited to this embodiment. The suction device 40 may be any device that can draw the cells 3. For example, the suction device 40 may be a blower, an aspirator, or a pump.

Modifications will be described below.

### First Modification

FIG. 11 is a schematic cross-sectional view of a portion of a filter of a first modification. Referring to FIG. 11, a top portion 14A of the projection 13A in the first modification may have any shape where the thickness of the projection 13A decreases toward the top portion 14A, and the top portion 14A is not necessarily pointed. For example, the top portion 14A of the projection 13A may have a substantially trapezoidal shape or a semicircular shape. Even with such a configuration, the cell membranes 4 can be efficiently disrupted.

### Second Modification

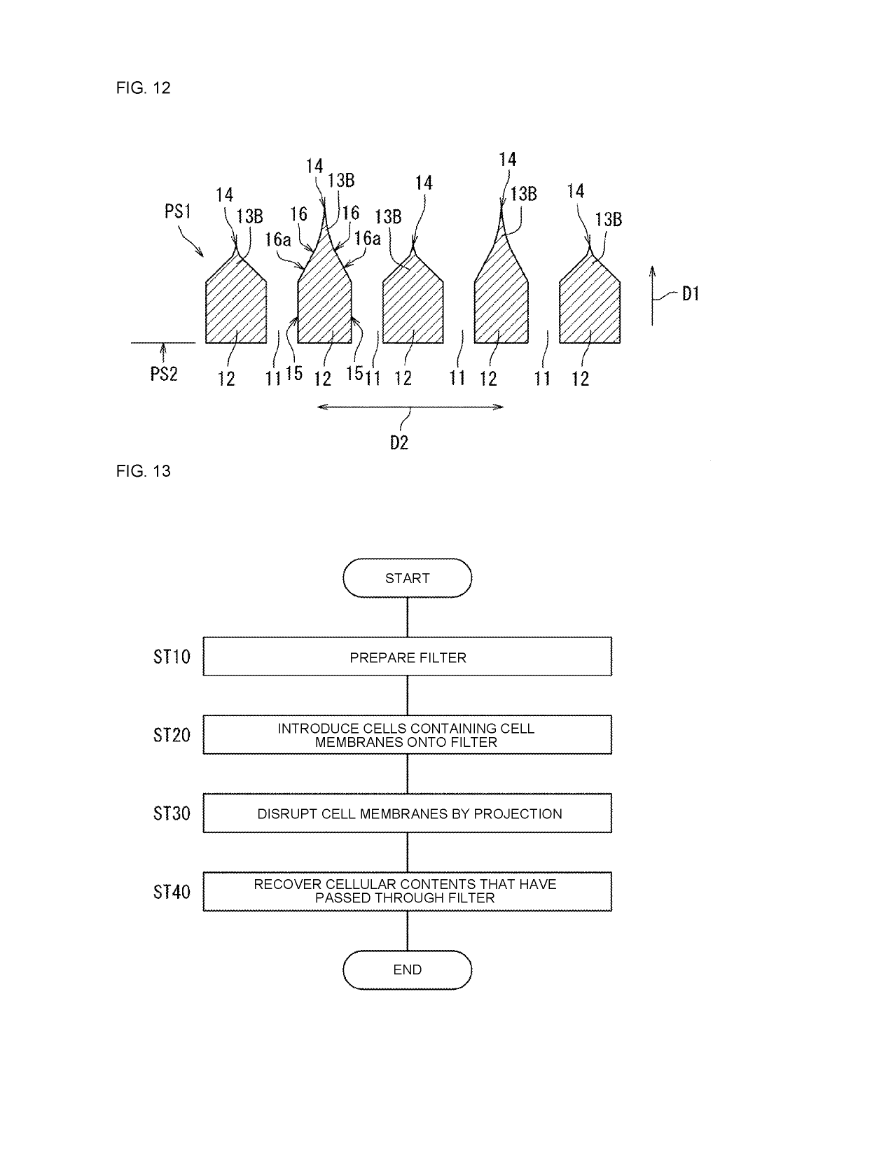

FIG. 12 is a schematic cross-sectional view of a portion of a filter of a second modification. Referring to FIG. 12, a projection 13B in the second modification may have a flat inclined surface 16a, which is inclined toward the through-holes 11, in at least part of the outer surface 16 of the projection 13B. Accordingly, the projection 13B may be tapered. This configuration allows the cellular contents 5, which have been discharged outside the cell membranes 4, to move gently toward the through-holes 11 and can thus suppress damage to the cellular contents 5.

### Third Modification

FIG. 13 is a flowchart of an example of a method for disrupting the cell membranes 4 according to a third modification. Referring to FIG. 13, the method for disrupting the cell membranes 4 in the third modification includes a step ST40 of recovering the cellular contents 5 that have passed through the filter 1. For example, the cellular contents 5 that have passed through the filter 1 can be recovered by the container 30 disposed below the filter 1. This configuration facilitates recovery of the cellular contents 5 in the cell membranes 4.

The recovered cellular contents 5 can be used for analyses or measurements, such as genetic analysis, cancer testing, or measurement of gene concentration. Genetic analysis may involve decoding gene sequences using a sequencer. Cancer testing may be performed by exosome analysis. Measurement of gene concentration may involve acquiring information for use in calibration of real-time PCR.

The method for disrupting the cell membranes 4 in the third modification may include a step of analyzing the recovered cellular contents 5.

The method for disrupting the cell membranes 4 may also include a step of recovering the cell membranes 4 remaining on the first principal surface PS1 of the filter 1. The method for disrupting the cell membranes 4 may also include a step of analyzing the cell membranes 4 remaining on the first principal surface PS1 of the filter 1.

The disruption device 2 for the cell membranes 4 may include, for example, a vibration device that vibrates the filter 1. The vibration of the filter 1 may disrupt the cell membranes 4 passing through the through-holes 11 while reducing the friction at contact points between the cell membranes 4 and the pointed top portions 14a.

The vibration source of the vibration device in the disruption device 2 may include vibration by rotation, impact, or ultrasound. The frequency of the vibration source can be selected in the range of 1 kHz to 100 kHz. This can assist the disruption effect of the pointed top portions 14a.

Furthermore, the vibration device in the disruption device 2 may be positioned away from the filter 1 in the device as long as the vibration propagates to the filter 1. For example, if no vibration-damping material for blocking the vibration of the filter 1 is provided between the filter 1 and a culture container, the culture container may be vibrated.

### Fourth Modification

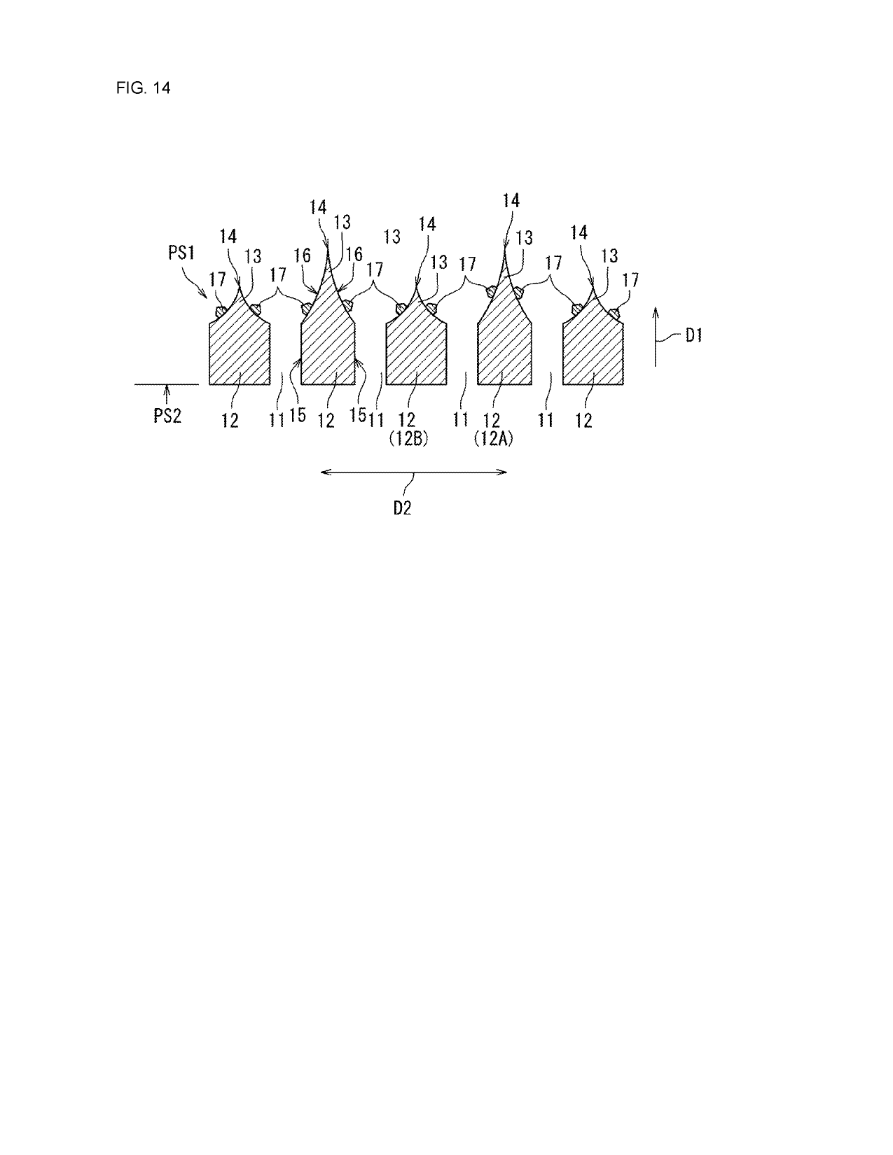

FIG. 14 is a schematic cross-sectional view of a portion of a filter of a fourth modification. Referring to FIG. 14, in the fourth modification, the projection 13 has a side projection 17 that projects from the outer surface 16. The side projection 17 may project in a direction different from the projection direction of the projection 13. For example, the side projection 17 may project in a direction intersecting the projection direction of the projection 13.

The shape of the side projection 17 may be pointed or rounded.

The side projection 17 can capture cells in which the cellular contents have been reduced as a result of disruption of the cell membranes by the projection 13, as well as the disrupted cell membranes, and can thus prevent resuspension of such cells and cell membranes. Since the cell membranes disrupted by the filter 1 are retained on the side projection 17, the cell membranes retained on the filter 1 can be directly analyzed with a microscope or another instrument to obtain information about the cell membranes.

### Example 1

In Example 1, an experiment was conducted in which the cell membranes 4 were disrupted using the filter 1 of the first embodiment. A 100 mL suspension of cells (HL-60) in a culture medium was prepared in a culture container. A 10 μL aliquot of the cell suspension was taken using a micropipette. The cell concentration was measured using Countess (AMQAF2000) available from Thermo Fisher Scientific and found to be 6 × 10⁵ cells/mL. The average cell size was 14 μm. A 1 mL aliquot was taken from the culture container using a micropipette and used as a sample (total cell count: 6 × 10⁵ cells).

The filter 1 was made of aluminum oxide. As viewed from the principal surface direction, the filter 1 had a substantially circular outer shape. The diameter of the filter 1 was measured at 8 locations using a caliper, and the average diameter was found to be 13.2 mm. The thickness of the filter 1 was measured at 8 locations using a caliper, and the average thickness was found to be 1.9 mm. The through-holes 11 and their surrounding region were observed in the principal surface direction of the filter 1 using a SEM (Scanning Electron Microscope). The hole size of eight through-holes 11 was measured using the SEM, and the average hole size was found to be 728 nm. When the through-holes 11 and their surrounding region were observed using the SEM in a direction inclined at 30 degrees to the first principal surface PS1, the projection 13 was found to surround the through-holes 11, and the top portion 14 of the projection 13 was pointed at multiple locations.

The filter 1 was placed at the inlet of a newly prepared 15-mL centrifuge tube, and 1 mL of the sample was dropped onto the first principal surface PS1 of the filter 1. The sample was retained on the first principal surface PS1 of the filter 1. Next, the inside of the 15-mL centrifuge tube was depressurized using a syringe connected to the 15-mL centrifuge tube. As a result, the liquid passed through the plurality of through-holes 11 from the first principal surface PS1 of the filter 1 to the second principal surface PS2 and into the 15-mL centrifuge tube. The syringe operation lasted for two seconds, and liquid permeation was observed within approximately two seconds.

The filter 1 was removed, and the first principal surface PS1 of the filter 1 was observed under a microscope. As a result, intact cells 3, fragments of disrupted cell membranes 4, and spherical cell nuclei were observed, indicating that the filter 1 had disrupted the cell membranes 4 of many cells 3. As a result of counting the number of intact cells by microscopic observation, six cells 3 were counted per 100 μm². This corresponded to 0.81 × 10⁵ cells when converted the area of the first principal surface PS1 of the filter 1. In other words, the cell membranes 4 of 86.5% of the cells 3 contained in the sample were successfully disrupted.

A mixed solution of 25 nM of the fluorescent dye MitoTracker Red FM in 1.5% DMSO was added to the liquid in the 15-mL centrifuge tube. After being left to stand at 37°C for 30 minutes, the resulting mixture was observed under a fluorescence microscope. As a result, a plurality of red spherical objects were observed, indicating that mitochondria, which are one type of the cellular contents 5, were successfully extracted. The size of the mitochondria was smaller than the hole size (average 728 nm) of the filter 1. In other words, the cellular contents 5 were successfully extracted.

### Example 2

In Example 2, an experiment was conducted in which the cell membranes 4 were disrupted using the filter 1 of the first embodiment, and the cellular contents 5 were extracted and analyzed. The procedure will be described below.

(1) An E. coli culture broth was vortexed for 5 seconds using a vortex mixer so as to obtain a uniformly dispersed culture broth.

(2) Using a micropipette, 1 mL of the E. coli culture broth vortexed in (1) was dispensed into a centrifuge tube.

(3) The E. coli culture broth was introduced onto the filter 1 of the first embodiment. In this step, the tip of the micropipette was moved during dropping so that the liquid uniformly wetted the entire principal surface of the filter 1.

(4) The system was left to stand for 3 minutes until the liquid on the first principal surface PS1 of the filter 1 had completely passed through.

(5) The filter 1 was removed from the container 30.

(6) The cell membranes 4 and the liquid containing the cellular contents 5 that had passed through the filter 1 were analyzed.

In Example 2, microscopic observation of the liquid containing the cellular contents 5 revealed that the nuclei did not aggregate and remained in an isolated state. Since the cellular contents 5 passed through the plurality of through-holes 11 of the filter 1 while being distributed into the through-holes, the cellular contents 5 were less likely to aggregate.

In Example 2, microscopic observation of the liquid containing the cellular contents 5 revealed that intracellular organelles retaining organelle contact sites that had been fluorescently labeled in advance were successfully extracted. The cellular contents 5 in the cell membranes 4 were successfully collected without chemical compositional changes.

In Example 2, microscopic observation, X-ray structural analysis, and infrared spectroscopy of the first principal surface PS1 of the filter 1 removed from the container 30 revealed the presence of membrane fragments retaining the mosaic structures or molecular arrangements of the cell membranes 4. The cell membranes 4 were successfully retained on the first principal surface PS1 of the filter 1.

In Example 2, X-ray structural analysis of the first principal surface PS1 of the filter 1 removed from the container 30 allowed analysis of the three-dimensional structure of cell membrane proteins. The cell membranes 4 were successfully retained on the first principal surface PS1 of the filter 1.

In Example 2, X-ray structural analysis of the first principal surface PS1 of the filter 1 removed from the container 30 revealed the presence of lipid rafts. Since the cell membranes 4 can be retained on the first principal surface PS1 of the filter 1 while protecting the intracellular environment (e.g., pH, or charge states used for signal transduction), the lipid rafts were successfully analyzed without disruption.

### Example 3

In Example 3, an experiment was conducted in which the cell membranes 4 were disrupted using the disruption device 2 including a vibration device that vibrates the filter 1. The procedure will be described below.

(1) A 100-mL suspension of floating cells (HL-60) in a culture medium was prepared, and the number of cells was measured using Countess (AMQAF2000) available from Thermo Fisher Scientific.

(2) The suspension in (1) was poured onto the filter 1 being vibrated by the vibration device.

(3) The number of cells from which cellular contents 5 had passed through the filter 1 was measured using Countess (AMQAF2000) available from Thermo Fisher Scientific.

(4) The cell disruption efficiency was calculated from the number of cells before passage through the filter 1 and the number of cells after passage through the filter 1.

In Example 3, the total number of cells and the disruption efficiency were compared with those in Example 2 in which vibration was not used for disrupting cells. In Example 2, the disruption efficiency was 60% to 80% (relative standard error: 14.8%), whereas in Example 3, the disruption efficiency improved to 65% to 95% (relative standard error: 5.5%).

In Example 3, a container 31 having the filter 1 at its bottom was prepared, and a culture medium and adherent cells (VERO) were added and cultured. As a result, the adherent cells were successfully retained on the first principal surface PS1 of the filter 1. Upon vibration of the container 31, the contents of the disrupted adherent cells flowed to the second principal surface PS2 of the filter 1, and membrane proteins constituting the cell membranes were observed from the cell membranes 4 remaining on PS1 without disruption of the arrangement of the cells.

In this description, terms such as "first" and "second" are used merely for purposes of description and should not be construed as indicating or implying relative importance or any ranking of technical features. A feature designated as "first" or "second" is intended to indicate or imply that one or more such features are included.

Although the present disclosure is fully described in connection with preferred embodiments with reference to the accompanying drawings, various modifications and alterations will be apparent to those skilled in the art. It should be understood that such modifications and alterations are within the scope of the present disclosure as long as they fall within the scope of the present disclosure defined in the appended claims.

The filter of the present disclosure is useful for applications of disrupting cell membranes or extracting cellular contents inside cell membranes.

### Reference Signs List

1 filter

2, 2A disruption device

3 cell

4 cell membrane

5 cellular contents

10 filter part

11 through-hole

12 filter base body

13 projection

14 top portion

14a pointed top portion

14b different top portion

15 end portion

16 outer surface

16a inclined surface

17 side projection

20 frame part

30 container

40 suction device

50 aluminum plate

51 first oxide film

52 recess

53 second oxide film

54 hole

55 cathode plate

1. A filter for disrupting cell membranes, the filter comprising:
a filter base body that has a first principal surface
having a projection and a second principal surface on an opposite side from the first principal surface, the filter base body defining a plurality of through-holes through which the first principal surface communicates with the second principal surface,
wherein the projection is located between adjacent
through-holes and projects in a first direction away from the filter base body as viewed from a first principal surface side,
in a cross-section cut in a second direction in which the
plurality of through-holes are adjacent to each other, the projection is located inward of an end portion of the filter base body located between the adjacent through-holes, and
a thickness of the projection in the second direction
decreases toward a top portion of the projection in the first direction.

2. The filter according to claim 1, wherein in the second direction, the projection has an outer surface that curves toward the end portion from a central region of the filter base body located between the adjacent through-holes.

3. The filter according to claim 2, wherein the projection has a flat inclined surface in at least part of the outer surface of the projection, the flat inclined surface being inclined toward the adjacent through-holes.

4. The filter according to claim 1, wherein
the filter base body is a first filter base body,
the plurality of through-holes are a first plurality of
through-holes,
the projection is a first projection that projects from
the first principal surface of the first filter base body, and
the filter further comprises:
a second filter base body that is different from the
first filter base body and is located between adjacent first through-holes of the first plurality of through-holes, the second filter base body having a third principal surface having a second projection and a fourth principal surface on an opposite side from the third principal surface, the second filter base body defining a second plurality of through-holes through which the third principal surface communicates with the fourth principal surface, and
a height of the first projection differs from a height of the second projection.

5. The filter according to claim 1, wherein the projection surrounds one or more through-holes of the plurality of through-holes as viewed from the first principal surface side of the filter base body.

6. The filter according to claim 1, wherein when a height of the projection is defined as a distance from the second principal surface of the filter base body to the top portion of the projection, the height of the projection continuously changes.

7. The filter according to claim 5, wherein as viewed from the first principal surface side of the filter base body, an area of a region surrounded by the top portion of the projection is larger than an opening area of the one or more through-holes.

8. The filter according to claim 1, wherein the projection has a pointed top portion.

9. The filter according to claim 8, wherein
when a region is drawn by connecting three virtual
straight lines each being in contact with outer peripheries of two adjacent through-holes among three adjacent through-holes of the plurality of through-holes such that the three through-holes are included in the region as viewed from the first principal surface side of the filter base body,
the pointed top portion is located within the region.

10. The filter according to claim 8, wherein when a height of the projection is defined as a distance from the second principal surface of the filter base body to the top portion of the projection, a height of the projection at the pointed top portion is greater than a height of the projection at a different top portion.

11. The filter according to claim 2, wherein the projection has a side projection that projects from the outer surface.

12. A cell membrane disruption device comprising the filter according to claim 1.

13. The cell membrane disruption device according to claim 12, further comprising a suction device that draws cells containing the cell membranes on the first principal surface side of the filter base body through the plurality of through-holes to a second principal surface side thereof.

14. The cell membrane disruption device according to claim 13, further comprising a container to which the filter is attached, the container, filer and suction device being arranged such that gas in the container is drawn at a position downstream of the filter by the suction device.

15. The cell membrane disruption device according to claim 12, further comprising a vibration device that vibrates the filter.

16. A cell membrane disruption method comprising:
preparing a filter including a filter base body that has
a first principal surface having a projection and a second principal surface on an opposite side from the first principal surface and that defines a plurality of through-holes through which the first principal surface communicates with the second principal surface;
introducing cells containing cell membranes onto a first
principal surface side of the filter base body of the filter; and
disrupting the cell membranes by the projection of the filter.

17. The cell membrane disruption method according to claim 16, further comprising recovering cellular contents that have passed through the filter.

18. The cell membrane disruption method according to claim 16, wherein the disrupting of the cell membranes further includes drawing the cells on the first principal surface side of the filter base body through the plurality of through-holes to a second principal surface side.

19. The cell membrane disruption method according to claim 18, the method further comprising:
attaching the filter to a container; and
drawing gas in the container at a position downstream of the filter.
